(19) United States
(12) Patent Application Publication
(10) Pub. No.: US 2026/0151530 A1
(43) Pub. Date: Jun. 4, 2026

(54) HYDROXYL-BASED SYSTEM FOR FILTERING AND DISINFECTING AIR AND LIQUIDS AND A METHOD OF USE

(71) Applicant: Effy SHAARABANY, Ramat Gan (IL)

(72) Inventor: Effy SHAARABANY, Ramat Gan (IL)

(21) Appl. No.: 19/454,283

(22) Filed: Jan. 20, 2026

Related U.S. Application Data
(63) Continuation-in-part of application No. PCT/IL2024/050770, filed on Aug. 3, 2024.
(60) Provisional application No. 63/530,642, filed on Aug. 3, 2023.

(51) Int. Cl. A61L 9/22; A61L 2/14; A61L 2/235; A61L 9/014; B01D 53/32; B01D 53/86; C02F 1/28; C02F 1/72

(52) U.S. Cl. CPC A61L 9/22; A61L 2/14; A61L 2/235; A61L 9/014; B01D 53/323; B01D 53/8612; B01D 53/8687; C02F 1/283; C02F 1/725; A61L 2202/11; A61L 2202/17; A61L 2209/14; A61L 2209/22; B01D 2258/0275; B01D 2258/06; B01D 2259/818; C02F 2303/04; C02F 2303/16; C02F 2305/023

(57) ABSTRACT

Hydroxyl-based system for disinfecting contaminated air/liquid and inhibiting contaminants and/or odors and/or gases from the air/liquid rapidly. The Hydroxyl-based system comprises a hydroxyl generator for generating hydroxyl radicals having an increased lifetime ranging between seconds to days and an activated material characterized by a multi-porous surface. The hydroxyl radicals and the contaminated air/liquid flow through the activated material to allow fast filtration and disinfection of said contaminated air/liquid, wherein the hydroxyl radicals have an increased lifetime required to reach the activated material and to adsorb with contaminant molecules into cavities of the activated material. In addition, the hydroxyl radicals clean/renew the activated material and enhance the actions of the activated material. Thereby, the hydroxyl-based system disinfects the contaminated air/liquid and inhibits contaminants and/or odors and/or gases from said air/liquid rapidly.

### CROSS-REFERENCE TO RELATED PUBLICATIONS

This application is a Continuation-in-Part of International PCT Patent Application No. PCT/IL2024/050770 having International filing date of Aug. 3, 2024, which claims the benefit of priority of U.S. Provisional Patent Application No. 63/530,642, filed Aug. 3, 2023. All of these documents are incorporated by reference in their entirety.

### FIELD OF THE INVENTION

The present invention relates to a system and method for filtering and disinfecting air and liquids. More specifically, the present invention relates to a highly efficient system and method for rapid air/liquid filtration and disinfection.

### BACKGROUND OF THE INVENTION

Hydroxyl radicals (OH*) are oxidants that hold a neutral charge compared to the hydroxyl groups (—OH or HO—), also known as hydroxyl ions, that hold a negative charge.

Hydroxyl Radicals are highly efficient in disinfecting air, water and surfaces. This molecule is often referred to as the atmosphere's “detergent”, as it reacts with a large number of pollutants to start the purification process. It also plays an important role in eliminating Greenhouse Gases, such as Carbon Dioxide and Methane.

When used in small concentrations, Hydroxyl Radicals (OH*), can be employed to carry out hygienic functions against viruses, bacteria, allergens and mould, in order to erode any organic compounds found in the air and turn them into minerals or harmless water-soluble organic compounds.

The use of Hydroxyl Radicals can quickly reduce or eliminate odors (including human odors), volatiles, bacteria, and contaminates on a person or his or her clothing and equipment and in the space between the person and an animal that may be alerting wildlife to the presence of a human.

Since a closed space must be filled with hydroxyl in order to filter and disinfect the air, and in order for the hydroxyl to be effective, the closed space must be filled with it for a relatively long contact time (approximately 30 minutes compared to ozone which requires a few seconds), it is an aim of the present invention to provide an improved, a highly efficient, environmentally friendly Hydroxyl-based system for fast filtering and disinfecting air.

It is an aim of the present invention to provide a system for filtering and disinfecting air without the production of Ozone or other hazardous emissions.

### SUMMARY OF THE INVENTION

The present invention is of a hydroxyl-based system for filtering and disinfecting air and various liquids including water. Since this system does not produce ozone or hazardous emissions during its operation as a byproduct, it is much more environmentally friendly than other air/liquid purification products available today.

In accordance with some embodiments of the present invention, there is thus provided a hydroxyl-based system for disinfecting contaminated air/liquid and inhibiting contaminants and/or odors and/or gases from said air/liquid rapidly. The hydroxyl-based system comprising:
a hydroxyl generator for generating hydroxyl radicals, said hydroxyl radicals having an increased lifetime ranging between seconds to days, and an activated material characterized by a multi-porous surface;
wherein the hydroxyl radicals and said contaminated air/liquid flowing through said activated material to allow a fast filtration and disinfection of said contaminated air/liquid;
wherein said hydroxyl radicals having the increased lifetime required to reach the activated material and to adsorb with contaminant molecules into cavities of the activated material,
wherein said hydroxyl radicals cleaning/renewing the activated material and enhancing the actions of the activated material,
thereby, said hydroxyl-based system disinfecting the contaminated air/liquid and inhibiting contaminants and/or odors and/or gases from said air/liquid rapidly.

Furthermore, in accordance with some embodiments of the present invention, the hydroxyl radicals in the vicinity of said activated material requiring a contact time of less than one second with the contaminated air/liquid to disinfect the air and to inhibit contaminants and/or odors and/or gases from said air/liquid.

Furthermore, in accordance with some embodiments of the present invention, the hydroxyl generator comprises a casing having an air inlet at one end of the casing and an air outlet at the other end of the casing, electrostatic means comprising at least one spiked surface, said at least one spiked surface inducing corona discharge and/or cold plasma when high voltage is applied, said corona discharge and/or said cold plasma producing ozone molecules,
at least one energy source for producing rays, said at least one energy source is inter-displaced within said electrostatic means to have the produced rays in close proximity to said electrostatic means for maximizing said at least one energy source efficiency in converting said ozone molecules to hydroxyl radicals, whereby said hydroxyl generator is highly efficient in converting the ozone molecules produced to hydroxyl radicals.
Furthermore, in accordance with some embodiments of the present invention, the hydrogen generator producing ozone molecules and converting said ozone molecules to hydroxyl radicals OH*, said hydroxyl radicals OH* forming HO2 and/or H2O2 radicals having a lifetime of seconds to days, said hydroxyl generator has an efficiency of up to 100% in converting said ozone molecules into hydroxyl radicals.

Furthermore, in accordance with some embodiments of the present invention, a reciprocating process of said hydroxyl radicals OH* producing hydroperoxyl radicals HO₂ and/or H₂O₂ and said hydroperoxyl radicals HO₂ and/or H2O2 producing hydroxyl radicals OH* back and forth, thus, the hydroxyl radicals OH* having an increased lifetime and lasting for seconds to hours and even days.

Furthermore, in accordance with some embodiments of the present invention, the hydroxyl radicals generated by the hydroxyl generator is combined with the contaminated air/liquid prior to flowing through the activated material.

Furthermore, in accordance with some embodiments of the present invention, the hydroxyl radicals generated by the hydroxyl generator and the contaminated air/liquid each flowing through the activated material.

Furthermore, in accordance with some embodiments of the present invention, the activated material is selected from activated carbon, zeolite, and a combination thereof.

Furthermore, in accordance with some embodiments of the present invention, the activated carbon is selected from activated coal and activated charcoal.

Furthermore, in accordance with some embodiments of the present invention, the hydroxyl radicals making multiple contacts with contaminant molecules on surfaces of said activated material and decomposing said contaminant molecules.

Furthermore, in accordance with some embodiments of the present invention, both the hydroxyl radicals and the contaminant molecules adsorbing into same space/cavity to allow a catalytic reaction inside a confined space of the porous surfaces.

Furthermore, in accordance with some embodiments of the present invention, flowing the hydroxyl radicals through the activated material increasing the effect of the hydroxyl radicals in filtering and disinfecting the contaminated air/liquid.

Furthermore, in accordance with some embodiments of the present invention, the activated material is activated as the hydroxyl radicals flowing through it, removing impurities and producing fine porous granules.

Furthermore, in accordance with some embodiments of the present invention, the hydroxyl-based system reducing the amount of said activated material required for filtering and disinfecting said air/liquid.

Furthermore, in accordance with some embodiments of the present invention, the hydroxyl-based system allowing a reduced contact time of the air/liquid with the hydroxyl radicals and the activated material to disinfect and inhibit contaminants and/or odors, and/or gases.

Furthermore, in accordance with some embodiments of the present invention, the hydroxyl-based system reducing the frequency of replacing the activated material and thus reducing the amount of waste.

Furthermore, in accordance with some embodiments of the present invention, the hydroxyl-based system requiring a contact time of less than one second to purify air/liquid with a flow rate of about 5,000 cubic meters per hour.

Furthermore, in accordance with some embodiments of the present invention, the hydroxyl-based system cleaning/renewing the activated material and enhancing the actions of the activated material.

Furthermore, in accordance with some embodiments of the present invention, the activated material is a non-toxic waste containing harmful waste materials and therefore does not have to be buried underground.

Furthermore, in accordance with some embodiments of the present invention, there is also provided a method for disinfecting air and inhibiting contaminants and/or odors and/or gases from air/liquid rapidly comprising:
(a) providing the hydroxyl-based system described above,
(b) generating hydroxyl radicals characterized by an increased lifetime ranging from seconds to days,
(c) either flowing a stream of the hydroxyl radicals and a stream of contaminated air/liquid in the vicinity of activated material or flowing a single stream of the hydroxyl radicals and the contaminated air in the vicinity of activated material,
(d) allowing the hydroxyl radicals to make multiple contacts with the contaminant molecules on the surface of the activated material and decompose the contaminant molecules, and
(e) allowing the hydroxyl radicals to clean/renew the activated material and enhance the actions of the activated material.
(F) (EPR) TECHNIQUE

### BRIEF DESCRIPTION OF THE FIGURES

FIG. 1 illustrates a hydroxyl-based system for filtering and disinfecting air/liquids such as water in accordance with some embodiments of the present invention.

FIG. 2 illustrates an alternative hydroxyl-based system for filtering and disinfecting air/liquid in accordance with some embodiments of the present invention.

FIG. 3 illustrates a method for filtering and disinfecting air/liquid in accordance with some embodiments of the present invention.

FIG. 4 illustrates quartet EPR signals attributed to OH trapping by DMPO (left) and BMPO (right).

FIG. 5A illustrates increase in BMPO-OH signal after 3 min bubbling with no scavenger.

FIG. 5B illustrates increase in BMPO-OH signal after 3 min bubbling with DMSO scavenger.

FIG. 6 illustrates increase of BMPO-OH signal with increase in bubbling time from 1 to 6 min.

### DETAILED DESCRIPTION OF THE FIGURES

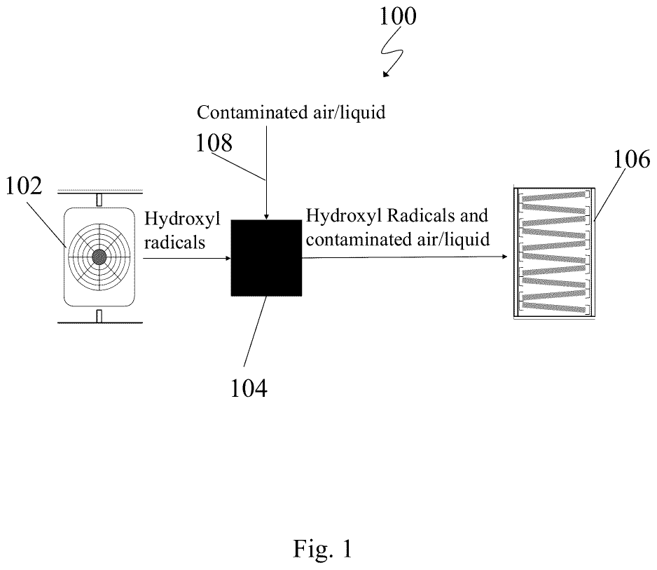

FIG. 1 illustrates a hydroxyl-based system 100 for filtering and disinfecting air/liquid such as water in accordance with some embodiments of the present invention.

The hydroxyl-based system 100 comprises a hydroxyl generator 102 and activated material 106, which is an adsorbent (adsorbing material), such as activated carbon, zeolite a combination thereof and the like where activated carbon may be activated coal, activated charcoal and the like.

Specifically, as seen in FIG. 1, the hydroxyl-based system 100 comprises a hydroxyl generator 102, a first container 104 and a second container containing the activated material 106.

In accordance with some embodiments of the present invention, hydroxyl radicals generated in hydroxyl generator 102 are transferred via a first conduit 110 to the first container 104 where they combine with contaminated air/liquid flowing in through a second conduit 108. The contaminated air/liquid and the hydroxyl radicals are then flowing via a third conduit 112 into the second container containing the activated material 106 where the hydroxyl radicals (1) make multiple contacts with the contaminant molecules on the surface of the activated material 106 and decompose the contaminant molecules, thus, disinfect the air/liquid and inhibit contaminants and/or odors and/or gases from the air/liquid rapidly, and (2) clean/renew the activated material 106 and enhance the actions of the activated material 106.

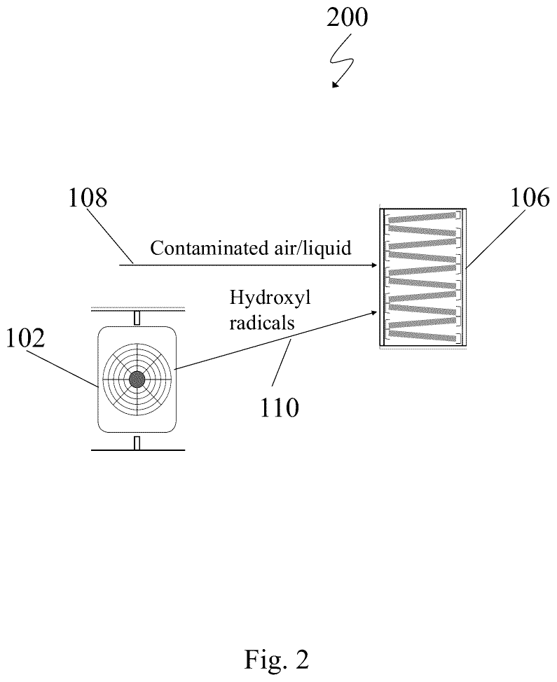

In accordance with some embodiments of the present invention, the contaminated air/liquid and hydroxyl radicals may be combined into a single mixed stream prior to entering the container containing the activated material 106 as shown and described below in FIG. 2.

FIG. 2 illustrates an alternative hydroxyl-based system 200 for filtering and disinfecting air/liquid in accordance with some embodiments of the present invention.

Specifically, as seen in FIG. 2, the hydroxyl-based system 200 comprises a hydroxyl generator 102 and a container containing an activated material 106.

In accordance with some embodiments of the present invention, separate streams of the hydroxyl radicals and contaminated air/liquid flow and meet in the container containing an activated material 106. That is, a stream of hydroxyl radicals generated by the hydroxyl generator 102 flow through conduit 110 and contaminated air/liquid flow through conduit 108 into a container containing an activated material 106 where the hydroxyl radicals (1) make multiple contacts with the contaminant molecules on the surface of the activated material 106 and decompose the contaminant molecules, thus, disinfect the air/liquid and inhibit contaminants and/or odors and/or gases from the air/liquid rapidly, and (2) clean/renew the activated material 106 and enhance the actions of the activated material 106.

In accordance with some embodiments of the present invention, the use of hydroxyl radicals with a multi-porous surface such as activated carbon, zeolite and the like, results in a faster rate of air/liquid filtration and disinfection than the use of hydroxyl radicals with contaminated air/liquid or using a multi-porous surface with contaminated air/liquid. The hydroxyl radical, being a powerful radical substance, gets the opportunity to make multiple contacts due to its passage along with various pollutants in a material with a multi-porous surface area such as activated carbon, zeolite and the like. The hydroxyl radicals use the activated material 106 as a receptacle for the impurities, that is, the hydroxyl radicals make contact with the impurities on the surface of the activated material 106 and decompose them.

The filtration and disinfection efficiency is related to the pore size of the porous surface. A porous surface with a larger pore size is able to adsorb both the hydroxyl radicals and the contaminant molecules into the same space/cavity, allowing a catalytic reaction to occur inside the confined space of the porous surface.

Thus, the transfer of the hydroxyl radicals through the activated material 106, a material with a porous surface, i.e., a large surface area and/or pore volume, increases the effect of the hydroxyl radicals in filtering and disinfecting the air passing therethrough, and therefore, there is no need to fill a cell/room with a hydroxyl radical as is done in existing technologies today.

Furthermore, during use, the activated material 106 is activated as the hydroxyl radicals flow through it, removing impurities and producing fine porous granules.

Thus, in accordance with some embodiments of the present invention, the hydroxyl-based system 100, 200 of the present invention, allows to:
significantly reduce the amount of the activated material required for the purification process, thus saving the energy required to transport a large amount of the activated material;
significantly reduce the contact time of the air/liquid with the activated material and with the hydroxyl;
significantly reduce the frequency of replacing the activated material and thus reduce the amount of waste that ends up in the landfill; and
clean/regenerate the activated material-since the hydroxyl radicals clean/regenerate the activated material, the waste will not be a toxic waste containing harmful waste materials and therefore will not have to be buried underground.

In accordance with some embodiments of the present invention, the hydroxyl generator 102 may be any hydroxyl generator and preferably the multifunctional air purification and sterilizing system of applicant's IL Patent No. 278580.

The multi-function air purifying and sterilizing system described in IL Patent No. 278580 comprises a casing with an air inlet at one end of the casing and an air outlet at the other end of the casing, electrostatic means consisting of at least one spiked surface. which induces corona discharge and/or cold plasma when high voltage is applied. The discharge of the corona and/or said cold plasma produces ozone molecules.

The multi-function air purifying and sterilizing system further comprises at least one energy source for producing rays, the at least one energy source is inter-displaced within said electrostatic means to have the produced rays in close proximity to said electrostatic means for producing ozone molecules and converting the ozone molecules to hydroxyl radicals.

The multi-function air purifying and sterilizing system producing ozone molecules convertible to hydroxyl radicals for disinfecting and inhibiting said biological contaminants and/or odors, and/or gases in the air stream and in the confined space.

In accordance with some embodiments of the present invention, the multi-function air purifying and sterilizing system described in IL Patent No. 278580 may be preferred due to its high efficiency in converting the ozone molecules produced to hydroxyl radicals.

The multi-function air purifying and sterilizing system described in IL Patent No. 278580 has an efficiency of up to 100% in converting said ozone molecules into hydroxyl radicals.

More specifically, the multipurpose air purification and sterilization system described in Patent No. IL 278580 produces ozone molecules and converts almost all ozone molecules to hydroxyl radicals. As a result of the highly efficient conversion of ozone molecules to hydroxyl radicals, i.e., in the absence or when a small number of ozone molecules are present, hydroperoxyl radicals HO2 or H2O2 may be formed (The process is detailed below in the experimental results).

Unlike OH* radicals which have a nanosecond lifetime, the hydroperoxyl radicals (oxidants) HO₂ or H₂O₂ which are produced from OH* are less reactive, and thus, have a longer lifetime of seconds to hours and even days (for H₂O₂), so they can diffuse around and convert back to the OH* radical as seen and explained below in the experimental data.

Such a reciprocating process of OH* radicals producing hydroperoxyl radicals (oxidants) HO₂ or H₂O₂ and hydroperoxyl radicals (oxidants) HO₂ or H2O2 producing hydroxyl radicals OH* back and forth is essential to the purification process of the present invention as described in FIGS. 1 and 2. More specifically, the hydroperoxyl radicals HO₂ and H₂O₂ have a long enough life time to reach the activated material 106 and continuously produce the hydroxyl radicals OH* as shown and explained below in the experimental data, and the hydroxy radicals OH* adsorb with the contaminant molecules into the cavities of the activated material 106.

Thus, as a result of the reciprocating process of OH* radicals producing hydroperoxyl radicals (oxidants) HO₂ or H₂O₂ and hydroperoxyl radicals (oxidants) HO₂ or H2O2 producing hydroxyl radicals OH* back and forth, the hydroxyl radicals OH* have an increased lifetime and can last for seconds to hours and even days.

In accordance with some embodiments of the present invention, the hydroxyl-based system 100, 200 of the present invention drastically reduces the time required to disinfect and inhibit contaminants and/or odors, and/or gases. That is, if the multi-function air purifying and sterilizing system described in IL Patent No. 278580 stands alone, the hydroxyl radicals need to be in contact with the contaminated air, the contact time, for about 30 minutes to purify (disinfect and inhibit contaminants and/or odors, and/or gases) air with a flow rate of about 5,000 cubic meters per hour.

In contrast, when using the hydroxyl-based system 100, 200 of the present invention, the hydroxyl radicals in the vicinity of an activated material need to be in contact with the contaminated air, a contact time of less than one second to purify (to disinfect and inhibit contaminants and/or odors, and/or gases) air having a flow rate of about 5,000 cubic meters per hour.

Thus, in accordance with some embodiments of the present invention, the advantage of the hydroxyl-based system 100, 200 is two-fold:
(1) the use of hydroxyl radicals with a multi-porous surface, activated material such as activated carbon, zeolite and the like, results in a faster rate of filtration and disinfection of air/liquids; and
(2) the hydroxyl radicals clean/renew the activated material and also enhance the actions of the activated material.

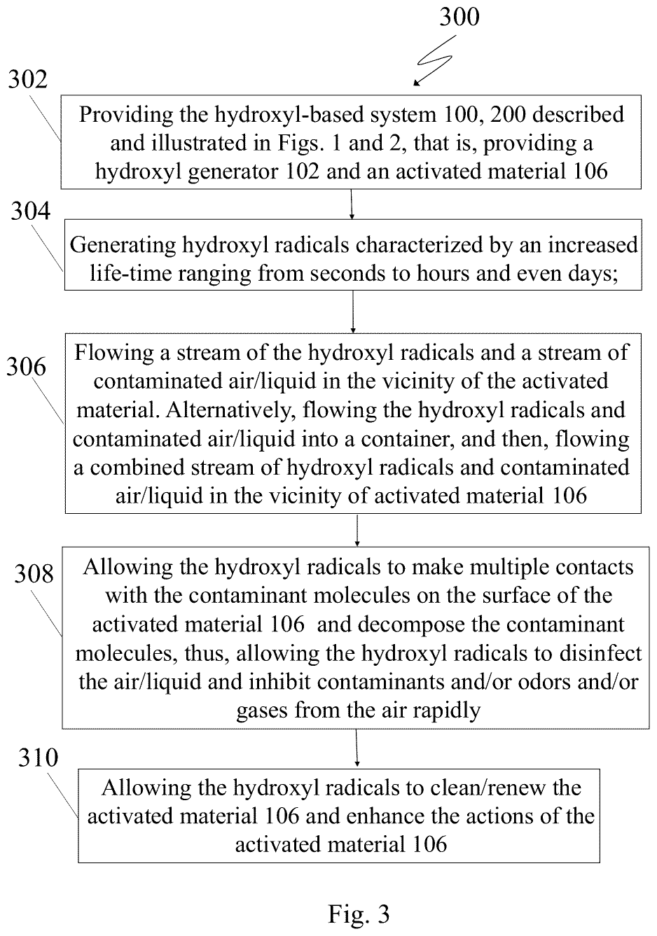

FIG. 3 illustrates a method for disinfecting air/liquid and inhibiting contaminants and/or odors and/or gases rapidly 300 in accordance with some embodiments of the present invention.

In accordance with some embodiments of the present invention, the method for filtering and disinfecting air/liquid 300 comprises the following stages:
Stage 302: Providing the hydroxyl-based system 100, 200 described and illustrated in FIGS. 1 and 2, that is, providing a hydroxyl generator 102 and an activated material 106;
Stage 304: Generating hydroxyl radicals having an increased life-time ranging from seconds to hours and even days;
Stage 306: Flowing a stream of the hydroxyl radicals and a stream of contaminated air/liquid in the vicinity of the activated material. Alternatively, flowing the hydroxyl radicals and contaminated air/liquid into a container, and then, flowing a combined stream of hydroxyl radicals and contaminated air/liquid in the vicinity of activated material 106;
Stage 308: Allowing the hydroxyl radicals to make multiple contacts with the contaminant molecules on the surface of the activated material 106 and decompose the contaminant molecules, thus, allowing the hydroxyl radicals to disinfect the air/liquid and inhibit contaminants and/or odors and/or gases from the air/liquid rapidly; and
Stage 310: Allowing the hydroxyl radicals to clean/renew the activated material 106 and enhance the actions of the activated material 106.

### (EPR) TECHNIQUE

### Experimental Results

### Hydroxyl Radical (OH) Generation in a Filtered Air Device Measured by the Electron Spin Technique (EPR) was Performed at Bar-Ilan University.

The presence of hydroxy radicals in water to which filtrated air, obtained from the multi-function air purifying and sterilizing system described in IL Patent No. 278580 was bubbled measured by using EPR spectroscopy technique coupled with spin trap.

In addition, a primary, indirect evidence shows existing of other reactive oxygen species such as H₂O₂/·O₂⁻ in such bubbled water.

By mimicking Fenton/Haber-Weiss reaction (Known reactions that produces OH radical by mixing H₂O₂/·O₂⁻ with Fe³⁺) the OH radical was generated just by mixing Fe³⁺ with bubbled water with no addition of H₂O₂, implying the presence of H₂O₂/·O₂⁻ already in such bubbled water.

A rough calculation of the OH amount, from EPR spectrum, was found to be 6 nM after 6 min bubbling which normalized to 10¹⁰ molecules per see per cm³.

This was calculated from comparing the intensity of the measured signal to that of a stable radical whose concentration is known.

Thus, the multi-function air purifying and sterilizing system described in IL U.S. Pat. No. 278,580 was used with a 1400 m³/hr capacity, featuring 2×16 W UVC lamps and an Electrostatic Precipitator for efficient performance.

Capable of collecting particles from 0.01 μm to 100 μm at 99% efficiency, the system handles high flow rates of up to 3,000,000 cfm (1400 m³/s) and particle loadings of 500 grams/m³. Energy-efficient, it operates at 16-100 Watts/1000 m³/h, generating Hydroxyl for comprehensive air purification.

With a power consumption of W850, the system is made of polished 1.5 mm stainless steel, including handles, hinges, and closures. Compact at 77 cm in height, 67 cm in width, and 57 cm in depth, it requires consumable replacements every 100 days for consistent performance.

### Direct Measurement Method—Using a Spin Trap to Measure OH Radical Coupled to the EPR Technique.

The OH radical is a reactive species that has a very short lifespan of about nano seconds. To overcome this challenge, a spin trap that catches the OH radical and create an adduct radical with a longer lifespan is used.

The two common and similar spin traps, DMPO and BMPO were used. DMPO has a half-life period of about 1-0.5 hours with OH, while BMPO has a half-life of a few hours with OH. The use of BMPO enabled repeatable measurements and reliable spectra.

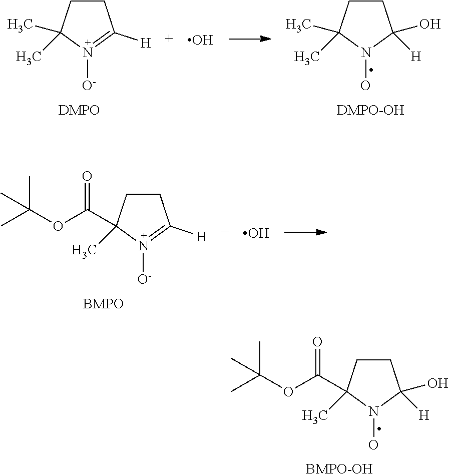

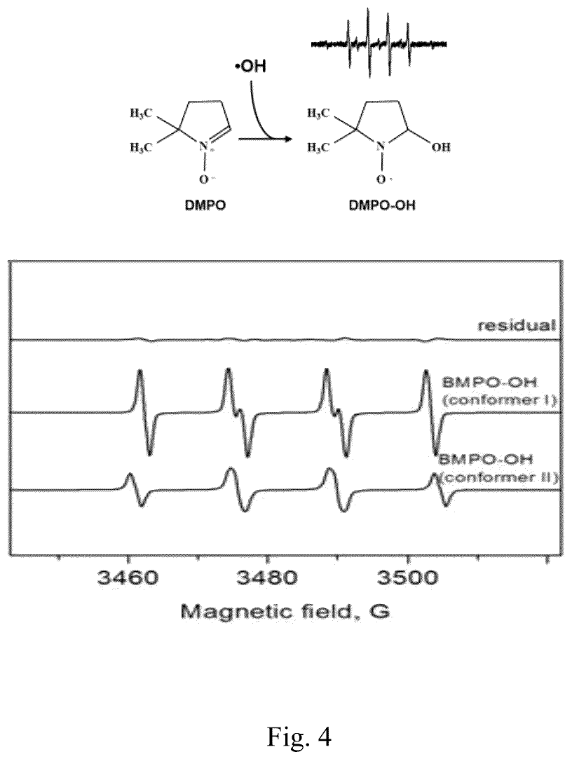

The spin adducts DMPO-OH and BMPO-OH have a characteristic EPR signal of Quartet, as can be seen in FIG. 4.

The DMPO was not sensitive enough and produced weak and not significant signal. However, when we used BMPO, we observed a consistent increase in the signal of BMPO-OH adducts after air bubbling. The BMPO-OH signal decreased when we added OH scavengers such as DMSO, EtOH, Iso-Propanol, or ascorbic acid. These suggest that the BMPO-OH signal attributed to trapping of OH radical by BMPO. Furthermore, when we increased the bubbling time, the BMPO-OH signal was also increased. All these results indicating that the OH radicals present in the water and originated from the bubbling.

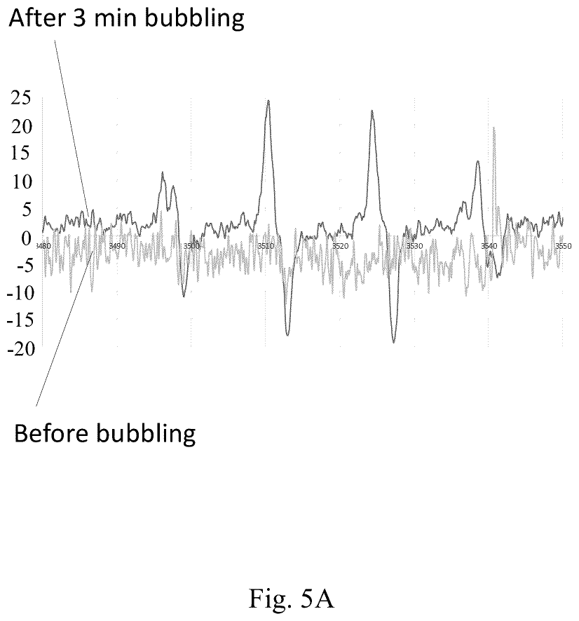

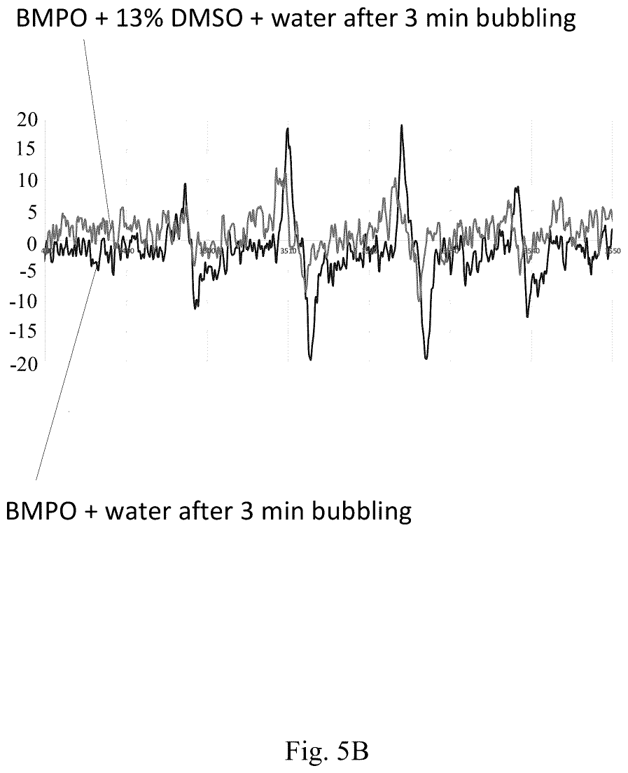

In FIGS. 5A-B and increase of BMPO-OH signal is observed after 3 min bubbling and decreasing of this increase with 13% DMSO (a specific OH scavenger).

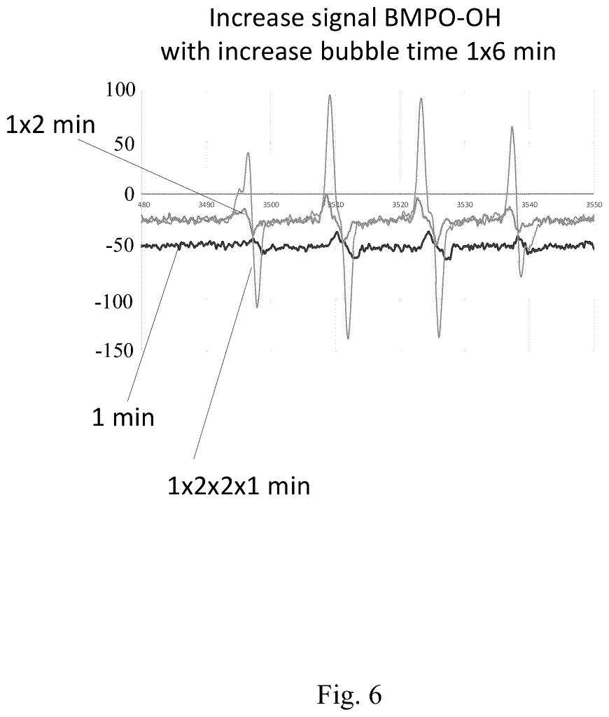

In FIG. 6 the increase in BMPO-OH signal with time can be observed.

### Suggested Pathway for OH Radical Generation in Bubbled Water

### 1) Generation of Oxygen Radical Inside the Device

### 1a) Generation of OH

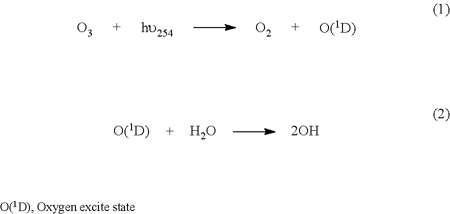

1b) Formation of Hydroperoxyl Radical (HO2)/Superoxide Radical (O2-) and Hydrogen Peroxide (H₂O₂) Following OH Generation.

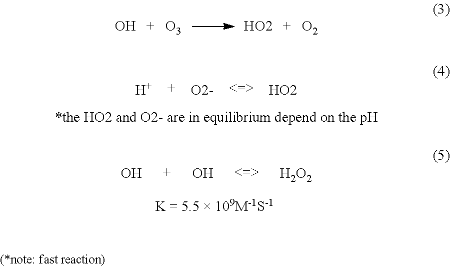

1C) Generation of OH from H2O2 Decomposition at 254 nM Irradiation

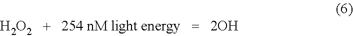

In addition to the generation of oxygen radicals by equation (1-6), concurrently, the accumulated radicals inside the device react continuously propagating producing additional radicals. Equation (7-11).

### 1d) Propagation Reaction

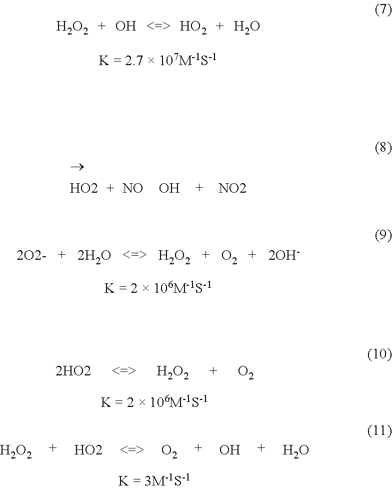

### 1e) Termination Reaction (Terminate Radical Reaction, Producing Non Radical Products)

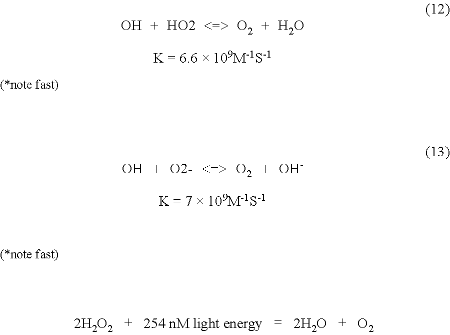

In such a mechanism, an abundance of oxygen radicals is produced which is amplified and weakened over time in a circular manner like a wave that extended along the device.

### 2) Generation of Oxygen Radical Outside the Device

Unlike the OH radicals that has nano sec life time thus will react before it will reach the exit port of the pipe device, the hydroperoxyl radicals HO2 or H₂O₂, have longer life time of second to hours and even days (for H₂O₂), thus they can diffuse around the exit port for a few centimeters and convert back the OH radical following the above equations.

Outside the device these radicals can react further with variety of compounds such as odor molecules (which are hydrocarbon molecules, oils) degrading the molecules.

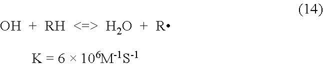

By using spin trap EPR technique, OH radical was measured in water which was bubbled with filtrated air originate from the device. The measurements were repeated several times with or without OH scavengers and prove the existing of OH radical.

Although OH is highly reactive, thus will react before it will reach the exit port of the pipe device, the hydroperoxyl radicals HO2 or H₂O₂, which produce from OH (equations 5, and 7 for example), are less reactive and their life time span from second to hours and even days (for H₂O₂). Therefore, they can diffuse around the exit port for a few centimeters and convert back the OH radical following equations, 8 and 11 for example.

The suggestion for the existing of other stable reactive species except from OH is based on literature known reactions (as shown here, equations 3-14) and on an experiment in which the Fenton/Haber-Weiss reaction is mimicked by replacing the H₂O₂ in the reaction with bubbled water.

The measured results give a good basis for the assumption that the mechanism of the device to neutralize bacteria/odors is based on the creation of reactive oxygen species.

### EXAMPLES

In accordance with some embodiments of the present invention, the hydroxyl-based system 100, 200 for filtering and disinfecting air has been tested and has produced remarkable results showing that gases have decomposed as well as pathogens and odors have disappeared.

In a first experiment, polluted air came out of a charcoal grill restaurant and there was a problem of bad smells.

The hydroxyl-based system 100 was situated in the flue line of the chimney.

The hydroxyl generators were placed in such a way that hydroxyl radicals were generated and mixed with the contaminated air before entering the coal zone. The contaminated air leaving the restaurant passed through the hydroxyl-based system 100 where the contaminated air particles came into contact with hydroxyl radicals for a few seconds, for example, about 3 seconds. The mixture of air and hydroxyl radicals then entered the coal zone and the exiting air stream was odorless. Thus, the hydroxyl-based system 100 produced excellent results and in addition the coal was renewed.

The hydroxyl generator was placed in such a way that hydroxyl radicals were formed and mixed with the contaminated air before entering the coal zone. The contaminated air leaving the restaurant passed through a hydroxyl-based system 100 where the contaminated air particles came into contact with hydroxyl radicals for a few seconds, for example, about 3 seconds. The mixture of air and hydroxyl entered the coal zone and the outgoing air stream was odorless. Thus, the system based on Hydroxyl 100 produced excellent results and in addition the coal was renewed.

Surprisingly, after turning off the system at night, the hydroxyl continued to form and reached the coal and slowly restored it.

In a second experiment, a sewage pit in a basement floor produced disturbing odors.

The hydroxyl-based system 100 was situated in the basement. The hydroxyl generator was placed in such a way that hydroxyl radicals were generated and mixed with the contaminated air before entering to the coal zone. The contaminated air in the basement was mixed with hydroxyl radicals and then passed through a coal zone. The odors were reduced though, not completely eliminated. There were still unpleasant smells in the ongoing air stream.

To further minimize/eliminate the odors in the air, an additional cartridge,

a 4 kg charcoal cartridge, of charcoal was added to the hydroxyl-based system 100. As a result, the odors dropped to zero, to the satisfaction of everyone who confirmed it.

It should be noted that it is known that odors of hydrogen sulfide can be reduced by activated carbon. However, with today's technology, about 500 or even 700 kg of coal is needed for every thousand cubic meters of air to significantly reduce such odors.

In contrast, the hydroxyl-based system 100, 200 of the present invention was used with about 4 kg of charcoal and completely reduced the odors of about fifteen hundred cubic meters of air.

1. Hydroxyl-based system for disinfecting contaminated air/liquid and inhibiting contaminants and/or odors and/or gases from said air/liquid rapidly comprising:
a hydroxyl generator for generating hydroxyl radicals, said hydroxyl radicals having an increased lifetime ranging between seconds to days, and an activated material characterized by a multi-porous surface;
wherein the hydroxyl radicals and said contaminated air/liquid flowing through said activated material to allow a fast filtration and disinfection of said contaminated air/liquid;
wherein said hydroxyl radicals having the increased lifetime required to reach the activated material and to adsorb with contaminant molecules into cavities of the activated material,
wherein said hydroxyl radicals cleaning/renewing the activated material and enhancing the actions of the activated material,
thereby, said hydroxyl-based system disinfecting the contaminated air/liquid and inhibiting contaminants and/or odors and/or gases from said air/liquid rapidly.

2. The hydroxyl-based system of claim 1, wherein the hydroxyl radicals in the vicinity of said activated material requiring a contact time of less than one second with the contaminated air/liquid to disinfect the air and to inhibit contaminants and/or odors and/or gases from said air/liquid.

3. The hydroxyl-based system of claim 1, wherein the hydroxyl generator comprises a casing having an air inlet at one end of the casing and an air outlet at the other end of the casing, electrostatic means comprising at least one spiked surface, said at least one spiked surface inducing corona discharge and/or cold plasma when high voltage is applied, said corona discharge and/or said cold plasma producing ozone molecules,
at least one energy source for producing rays, said at least one energy source is inter-displaced within said electrostatic means to have the produced rays in close proximity to said electrostatic means for maximizing said at least one energy source efficiency in converting said ozone molecules to hydroxyl radicals, whereby said hydroxyl generator is highly efficient in converting the ozone molecules produced to hydroxyl radicals.

4. The hydroxyl-based system of claim 2, wherein said hydrogen generator producing ozone molecules and converting said ozone molecules to hydroxyl radicals OH*, said hydroxyl radicals OH* forming HO2 and/or H2O2 radicals having a lifetime of seconds to days, said hydroxyl generator has an efficiency of up to 100% in converting said ozone molecules into hydroxyl radicals.

5. The hydroxyl-based system of claim 4, wherein a reciprocating process of said hydroxyl radicals OH* producing hydroperoxyl radicals HO2 and/or H2O2 and said hydroperoxyl radicals HO2 and/or H2O2 producing hydroxyl radicals OH* back and forth, thus, the hydroxyl radicals OH* having an increased lifetime and lasting for seconds to hours and even days.

6. The hydroxyl-based system of claim 1, wherein the hydroxyl radicals generated by the hydroxyl generator is combined with the contaminated air/liquid prior to flowing through the activated material.

7. The hydroxyl-based system of claim 1, wherein the hydroxyl radicals generated by the hydroxyl generator and the contaminated air/liquid each flowing through the activated material.

8. The hydroxyl-based system of claim 1, wherein said activated material is selected from activated carbon, zeolite, and a combination thereof.

9. The hydroxyl-based system of claim 8, wherein said activated carbon is selected from activated coal and activated charcoal.

10. The hydroxyl-based system of claim 1, wherein said hydroxyl radicals making multiple contacts with contaminant molecules on surfaces of said activated material and decomposing said contaminant molecules.

11. The hydroxyl-based system of claim 1, wherein both the hydroxyl radicals and the contaminant molecules adsorbing into same space/cavity to allow a catalytic reaction inside a confined space of the porous surfaces.

12. The hydroxyl-based system of claim 1, wherein flowing the hydroxyl radicals through the activated material increasing the effect of the hydroxyl radicals in filtering and disinfecting the contaminated air/liquid.

13. The hydroxyl-based system of claim 1, wherein the activated material is activated as the hydroxyl radicals flowing through it, removing impurities and producing fine porous granules.

14. The hydroxyl-based system of claim 12, wherein said hydroxyl-based system reducing the amount of said activated material required for filtering and disinfecting said air/liquid.

15. The hydroxyl-based system of claim 12, wherein said hydroxyl-based system allowing a reduced contact time of the air/liquid with the hydroxyl radicals and the activated material to disinfect and inhibit contaminants and/or odors, and/or gases.

16. The hydroxyl-based system of claim 12, wherein said hydroxyl-based system reducing the frequency of replacing the activated material and thus reducing the amount of waste.

17. The hydroxyl-based system of claim 12, wherein said hydroxyl-based system requiring a contact time of less than one second to purify air/liquid with a flow rate of about 5,000 cubic meters per hour.

18. The hydroxyl-based system of claim 12, wherein the hydroxyl-based system cleaning/renewing the activated material and enhancing the actions of the activated material.

19. The hydroxyl-based system of claim 18, wherein the activated material is a non-toxic waste containing harmful waste materials and therefore does not have to be buried underground.

20. A method for disinfecting air and inhibiting contaminants and/or odors and/or gases from air/liquid rapidly comprising:
(a) providing the hydroxyl-based system of claims 1-19,
(b) generating hydroxyl radicals characterized by an increased lifetime ranging from seconds to days,
(c) either flowing a stream of the hydroxyl radicals and a stream of contaminated air/liquid in the vicinity of activated material or flowing a single stream of the hydroxyl radicals and the contaminated air in the vicinity of activated material,
(d) allowing the hydroxyl radicals to make multiple contacts with the contaminant molecules on the surface of the activated material and decompose the contaminant molecules, and
(e) allowing the hydroxyl radicals to clean/renew the activated material and enhance the actions of the activated material.
